(19) United States
(12) Patent Application Publication
(10) Pub. No.: US 2016/0058599 A1
(43) Pub. Date: Mar. 3, 2016

(54) Cuboid human spinal traction device

(71) Applicant: Theodore Eugene Basler, Hazelwood, MO (US)

(72) Inventor: Theodore Eugene Basler, Hazelwood, MO (US)

(21) Appl. No.: 14/469,019

(22) Filed: Aug. 26, 2014

(51) Int. Cl. A61F 5/042; A61F 5/04

(52) U.S. Cl. CPC A61F 5/042; A61F 5/04

(57) ABSTRACT

The cuboid is different from all other spinal traction devices because it: has no hard frame, strap, rope, pulley, harness or motor; is lightweight; contours to the shape of the legs for comfort; spreads out the pressure necessary for traction over more surface area of the body decreasing pressure points and increases time in traction; is adjustable from zero to maximum pull; is placed on the edge of a bed with the user lying comfortably on the bed; requires no electricity or energy from the user; uses a completely relaxed human body and weight from legs to power its leverage; pulls comfortably on the legs to apply traction to the entire spine; incorporates a sleeping bed to facilitate traction and supply resistance on a large area of the back of the head and upper body for increased comfort. Inflatable models are deflated, folded and placed in a drawer or luggage.

### BACKGROUND

### Field

The background of the invention is the field of spinal traction to decompress discs between the vertebrae of the spine for pain reduction for humans. When discs between the vertebrae are bruised, torn, ruptured, compressed or otherwise damaged and or bulging beyond their normal position between the vertebrae, nerves can be pressed on by the disc material causing pain, numbness or loss of functional abilities. When the human spine is placed in traction, the muscles of the back are stretched and the vertebrae are pulled apart slightly. When the vertebrae are farther apart, there is more space in between the vertebrae where the discs are located. This extra space induces a vacuum between the vertebrae which can suction the bulging disc material back in between the vertebrae and away from nerves. The human body can heal itself. If a damaged body part like a broken bone is put back together or back in place for a long enough time the self-healing mechanisms of the body repair the bone. The inventor's theory is that if a damaged part of the body such as a disc between the vertebrae is suctioned back in place for a long enough period of time, the body can repair and heal it in that position and away from nerves.

The problem with prior art: Many devices cause uncomfortable or painful pressure points on certain areas of the human body when being used because they contact small amounts of surface area of the human body when they apply force. Some devices cause the user to be inverted which causes uncomfortable blood pressure in the head of the user. Some devices require the user to use physical energy end exertion to make the device work. Some devices require electricity to run a motor. Some devices require the user to get into the device or to be attached to the device with straps and damps. Some devices require the user to have to get down on the floor to be able to use the device. Some devices take up large amounts of room and get in the way; many of these are not as easily accessible because they are stored in basements or rooms away from frequented and easily accessible rooms such as a bedroom. Some devices are heavy. Some devices are difficult to store, and transport. It is extremely difficult or impossible to transport some devices in luggage. Some devices are rigid. Some devices include ropes and pulleys. Some devices include heavy weights. Because of the inherent features of prior art, they cannot be or are not used as often as is needed.

### BRIEF SUMMARY

### Object—Why the Cuboid is Made

The cuboid is made to be more quickly and easily accessible for use when needed and to be used more often than prior art. It is made for use in the bedroom and on a sleeping bed for accessibility and comfort It is made lightweight to increase ease of use. It is made to apply the force that causes traction to be applied over larger areas of the human body reducing pressure points, increasing comfort and time in traction. As an inflatable it can easily be deflated by way of a large inside diameter valve, is pliable, can be faded and can be stored in small places as in a drawer or in luggage for travel. it can easily be stored out of the way in an easily accessible bedroom drawer and quickly inflated for use on a bed when needed.

### Advantages of the Cuboid, How it Solves Problems of Prior Art

There are many prior art devices in use providing traction for spinal decompression. Other devices provide less time in traction sessions because of discomfort from pressure points from their use or their inaccessibility because of storage or transportation problems. Because of the time restrictions from the features of prior art, traction and decompression from them are limited. There are no other prior art spinal traction devices close to or resembling the cuboid human spinal traction device.

Because of the inherent qualities of the cuboid, its use is ergonomic and the human body is comfortable, completely relaxed and pressure points are reduced. Because the pressure on the body from the pull from the cuboid that causes traction is spread out over more square inches of surface area of the body, longer traction sessions can more comfortably be had for more pain reduction and healing.

Other advantages of the cuboid human spinal traction device in comparison to other spinal fraction devices are: The cuboid does not have harnesses, straps, cables, ropes, belts, pulleys or anything that attaches to the human body. The user does not have to put on, get into, attach anything or take anything off. The cuboid is not rigid and has no hard frame. The cuboid is pliable and conforms to the shape of the human legs, The user of the cuboid is horizontal on the comfort of a sleeping bed as compared to being on the floor, a table, in a mechanical device or hanging upside down. The cuboid is more comfortable than other traction devices allowing it to be used more often and for longer periods of time. The cuboid can comfortably be slept on with traction applied as compared to most all other traction devices. The cuboid does not take up as much space when not in use as most other traction devices including inversion devices which makes the cuboid easier to store, more accessible and more likely to be used. The inflatable cuboid is lighter and smaller than most other devices and requires very little effort to put it in place for traction and it requires no effort to use as compared to other traction devices. The inflatable cuboid can easily be deflated, folded and transported in small luggage along with clothes for use away from home. The cuboid uses gravity, friction and resistance to produce force from leverage; the cuboid uses no motor or power source as compared to some other traction devices. The cuboid applies traction when and because the human body is completely relaxed as compared to some other traction devices that require the user to use muscle force or electricity to make the devices work. The amount of pull and traction from the cuboid is completely and easily adjustable from zero pull and traction up to maximum pull and traction for a sustained constant pull and traction while in use as compared to some other traction devices.

### BRIEF DESCRIPTION OF THE SEVERAL VIEWS OF THE DRAWINGS

FIGS. 1, 2, 3, 5, 7, 9 and 36 show unassembled basic parts of the cuboid models. FIGS. 3-8 and 29 show models with handles. FIGS. 13-22 show the sizes and size range of all models and the basic shape of cuboid models of any type of construction. FIGS. 12, 23, 27, 28, 32-35, 39-46, and 52-54 show cuboids with contour side(s) or face(s), FIGS. 1-12, 23-28, and 32-46 show inflatable, models with their parts, FIGS. 13-22, 39-46, and 52-42 show external shapes of models of any type of construction. FIGS. 11, 12, 23, 25, 30-46, 52-54 show models with premium features other than handles. FIGS. 39-46, and 52-54 show the external shape of foam models. FIGS. 49-51 show the cuboid in action. FIG. 47 shows a removable cover and FIG. 48 shows a sheet of anti-skid material.

### SPECIFICATION

### Detailed Description

There are multiple models of the cuboid human spinal traction device explained in the following text. The model being manufactured and offered for sale is described first. Following the manufactured model are descriptions of all models. This is followed by the Claim and the Abstract. Following the Abstract is a definition of the pads of the Figures and their corresponding alphabet indicators. The text is numbered on pages 1 to 32. Following the text are the Figures on pages numbered from 1 through 49. The 54 Figures are numbered 1 through 54.

### Model Being Manufactured and Offered for Public Sale

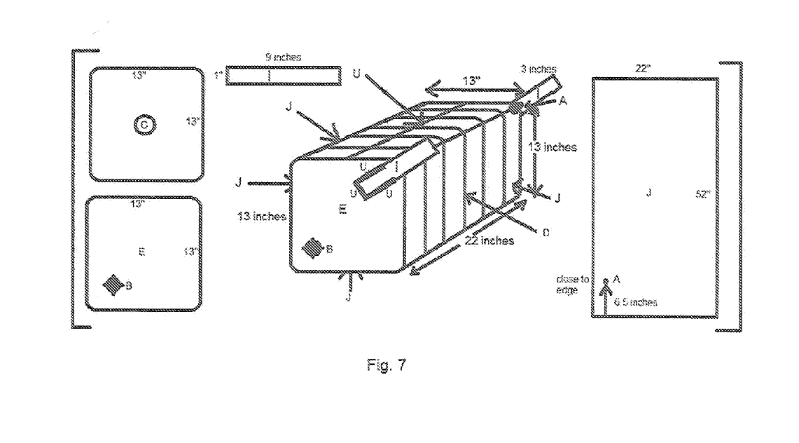

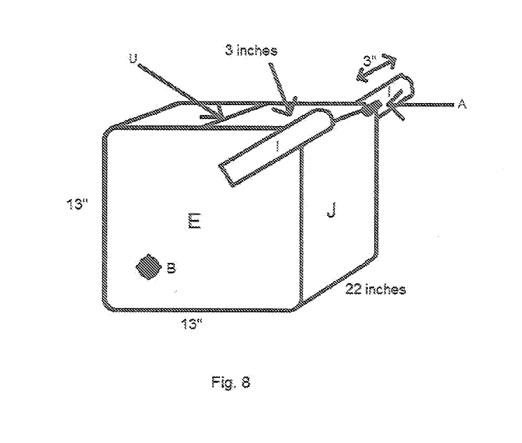

The model of cuboid being manufactured and offered for sale is a square cuboid and is shown in FIGS. 7 and 8. Its square cuboid dimensions are 22 inches×13 inches×13 inches. It is an inflatable. It has rounded corners. It has a large air inflation port.

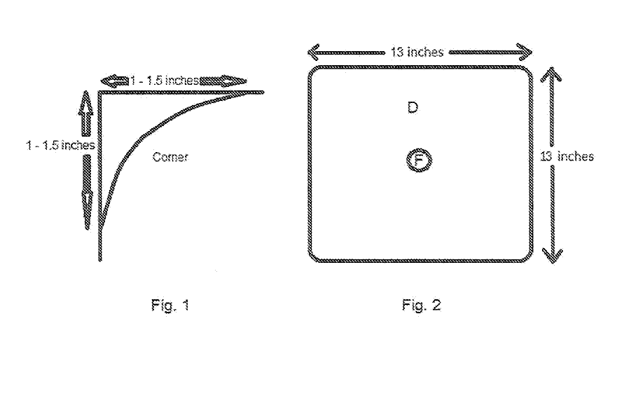

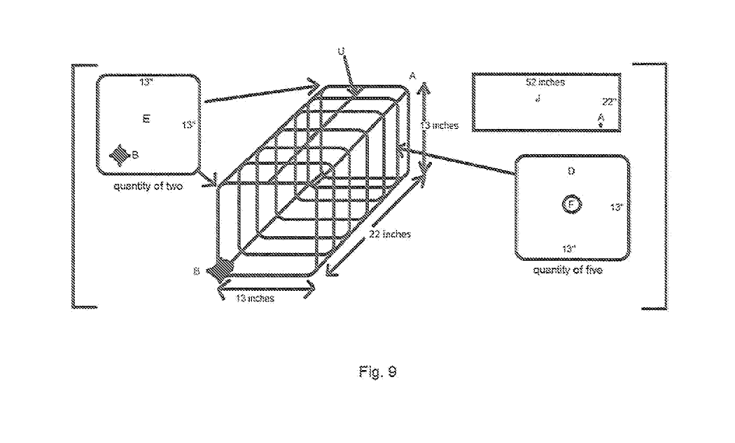

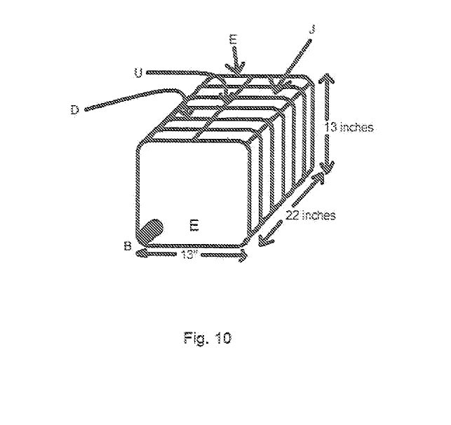

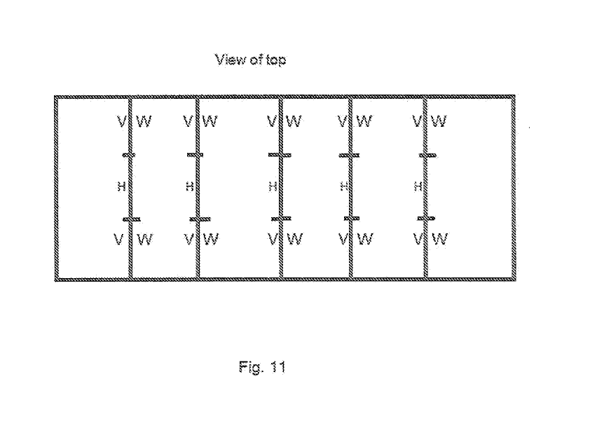

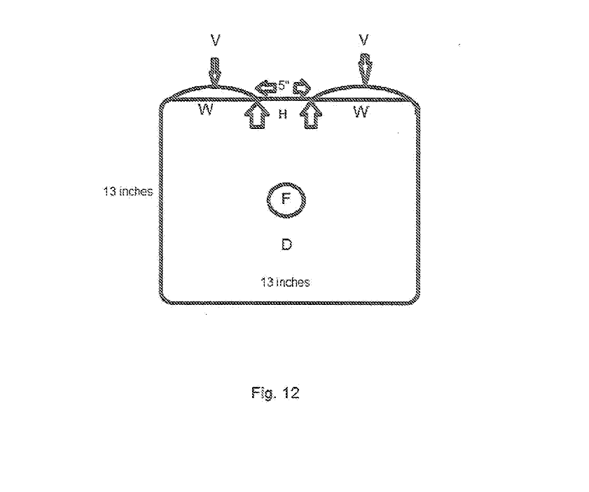

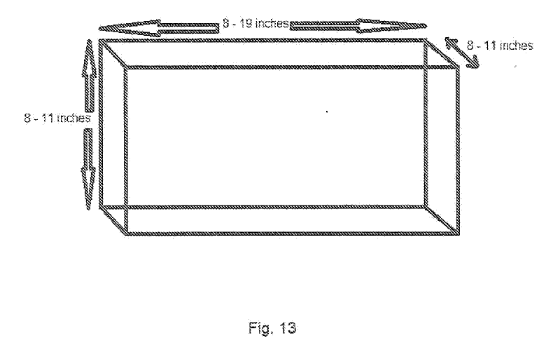

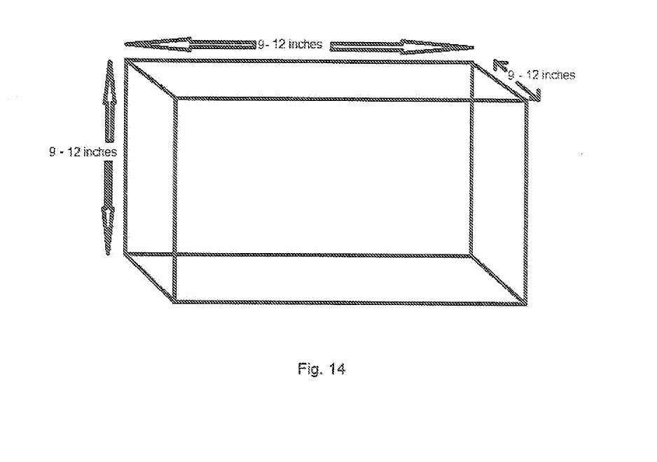

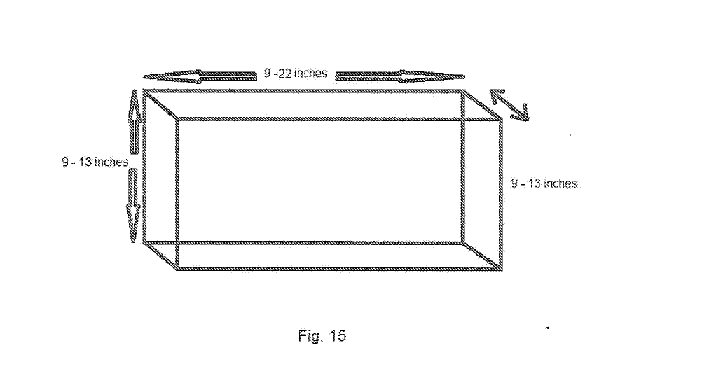

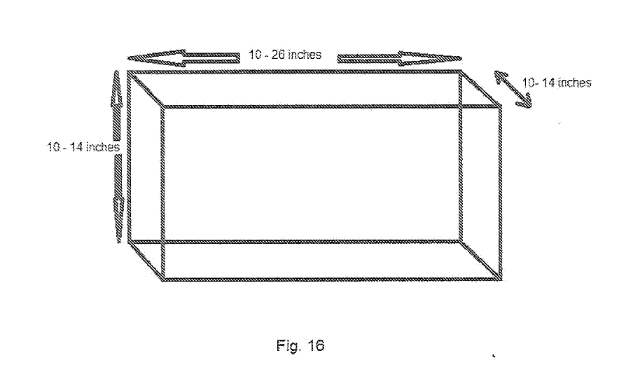

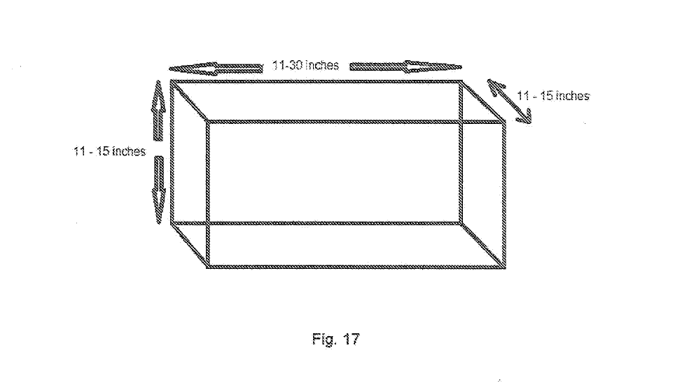

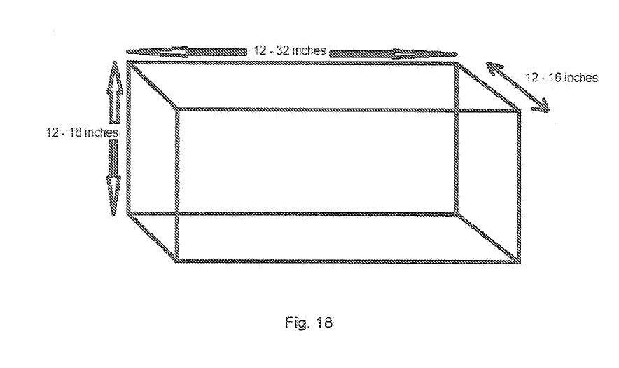

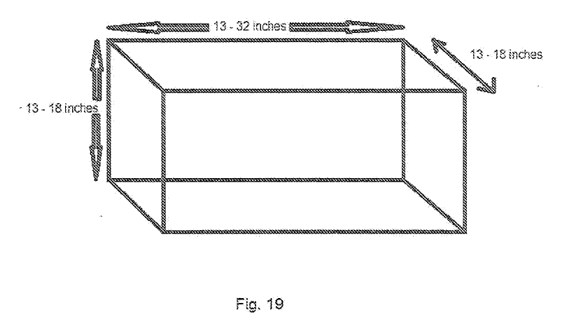

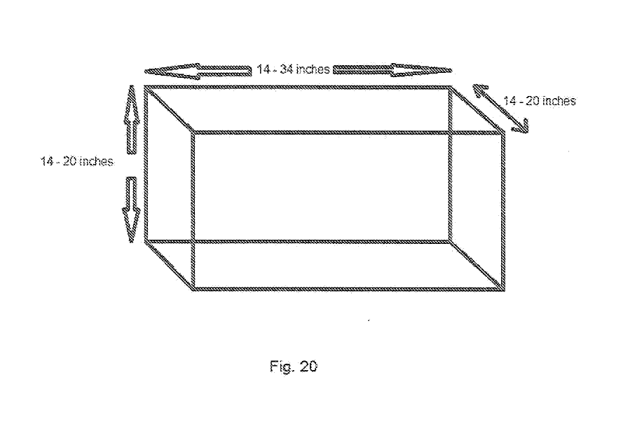

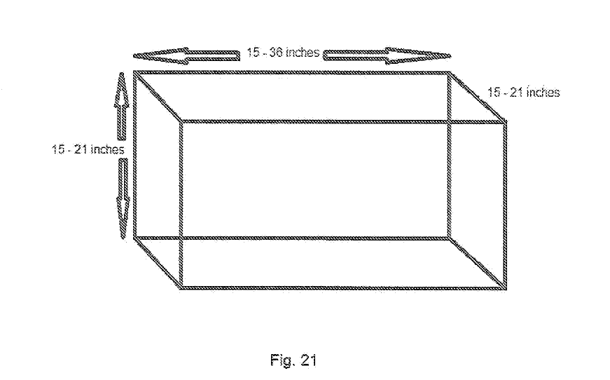

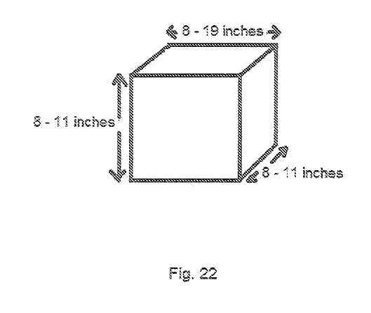

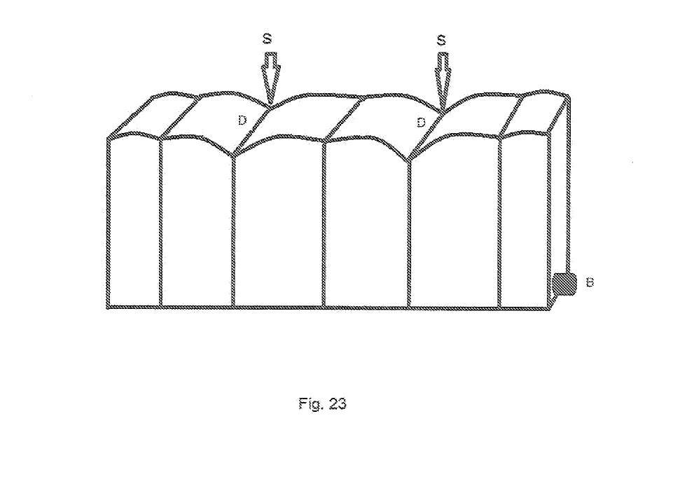

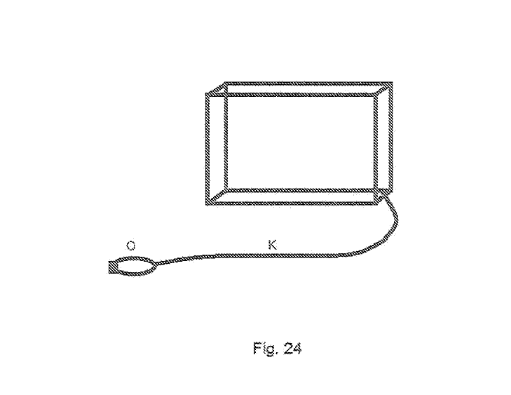

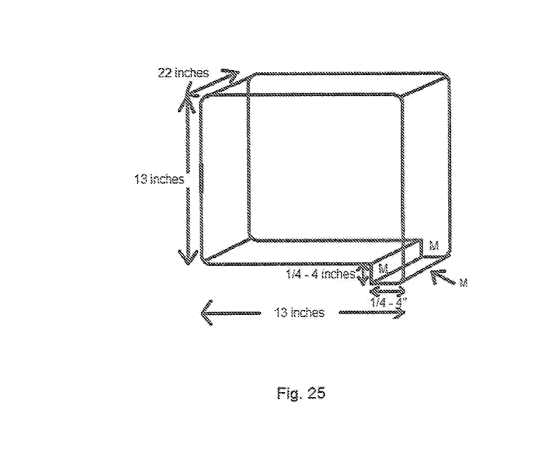

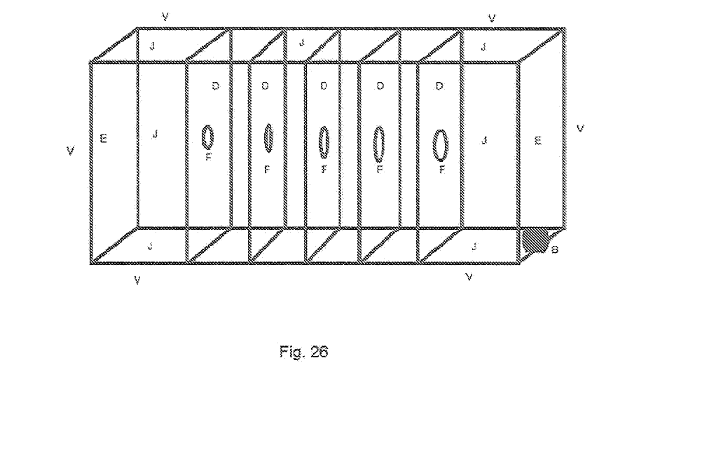

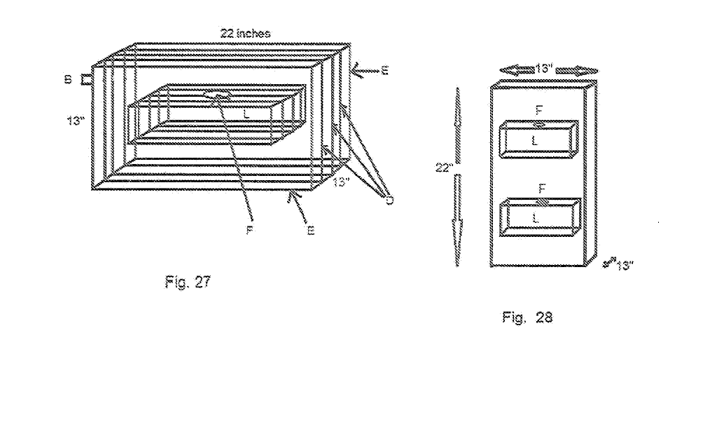

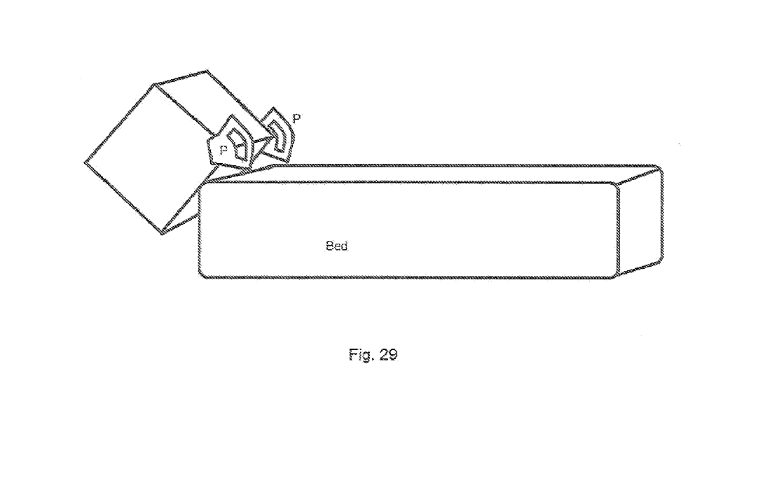

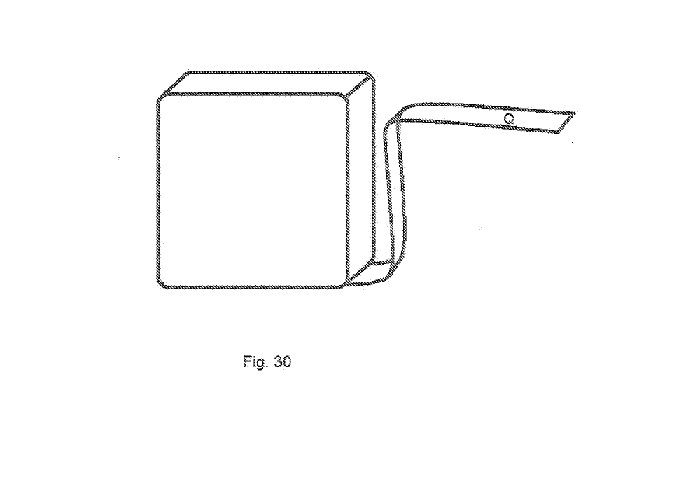

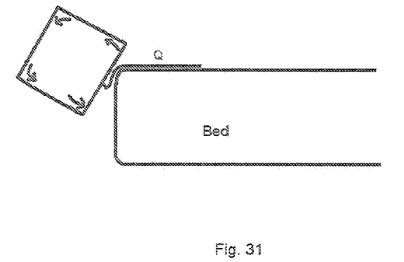

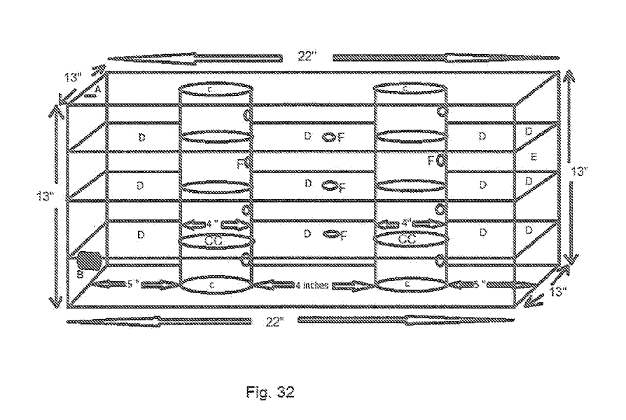

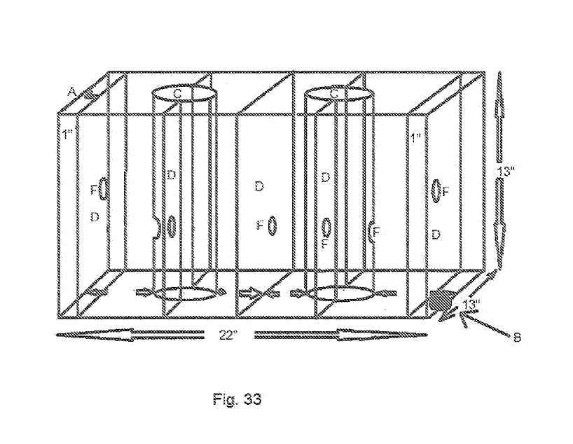

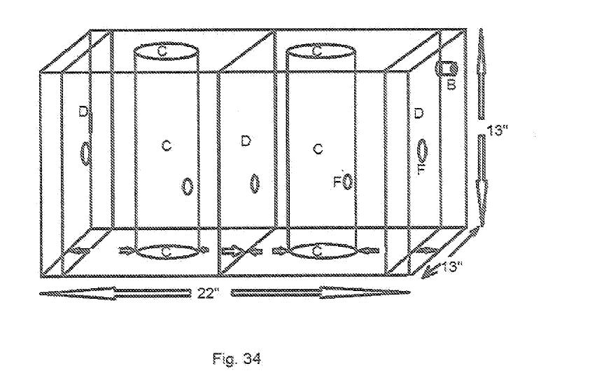

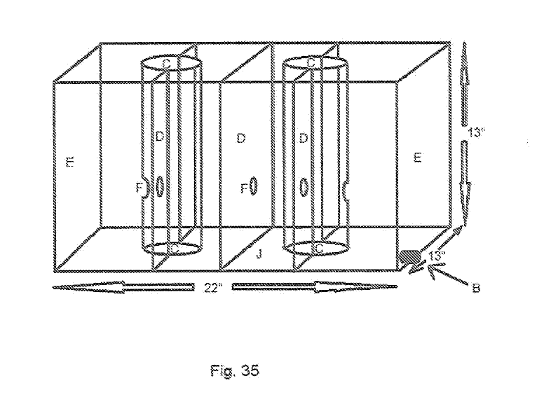

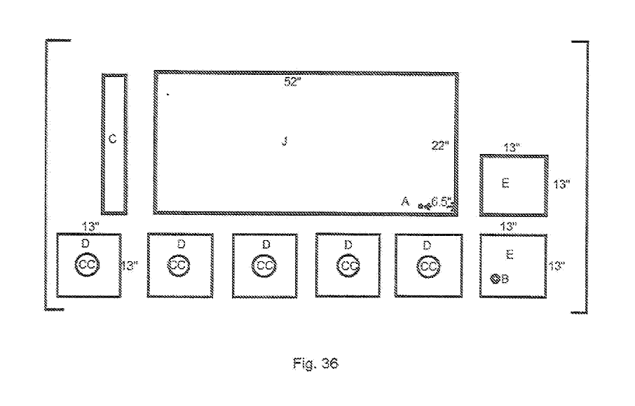

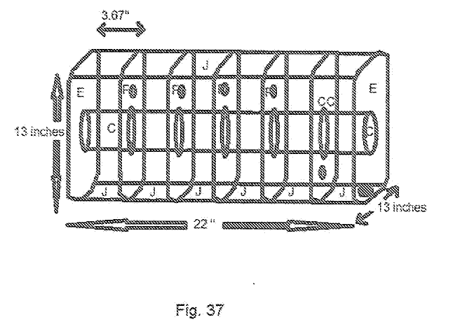

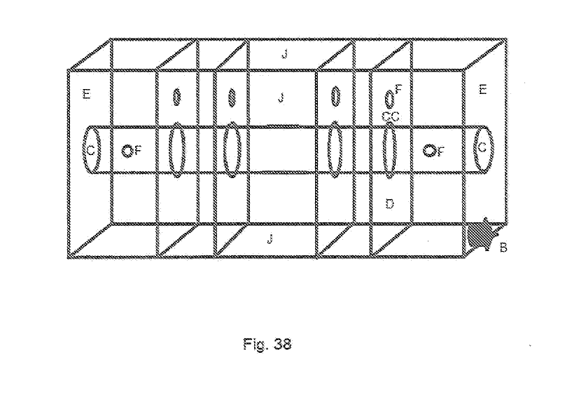

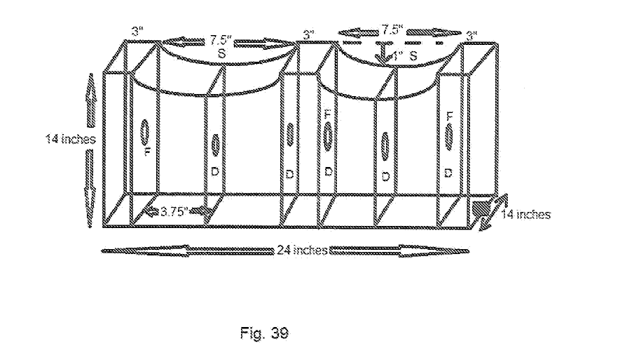

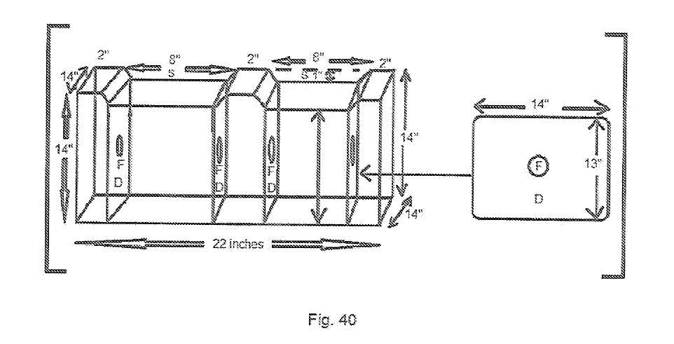

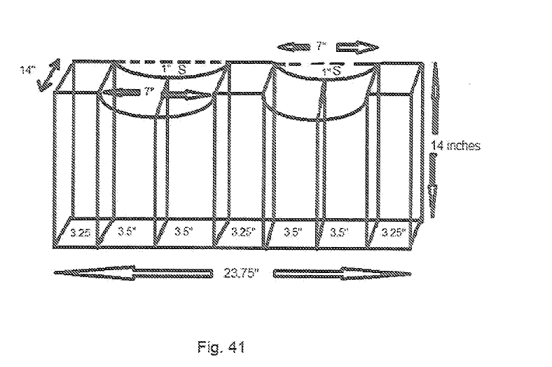

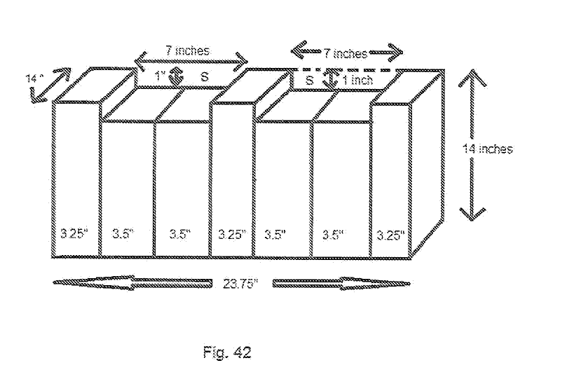

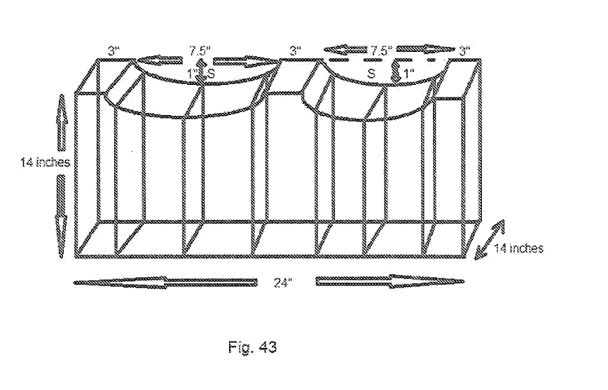

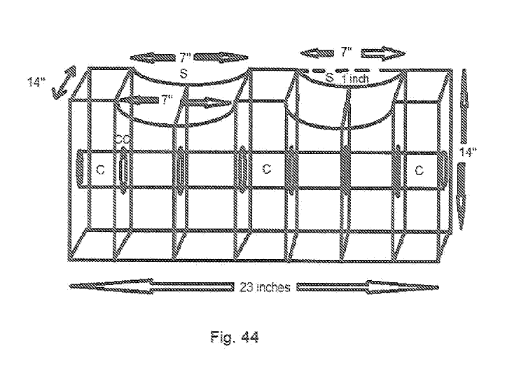

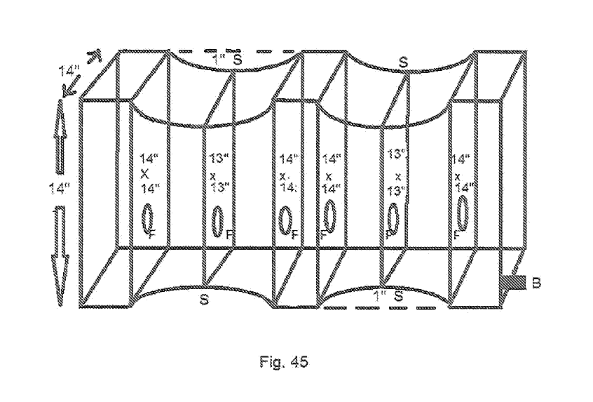

This cuboid is constructed of 0.45 millimeter thick poly vinyl chloride material FIGS. 1, 2 and 9 Parts D, E, and J. This pvc material has anti-skid qualities that help hold it in place while in use, The cuboid is constructed by radio frequency or high frequency welding (indicated by the letter U in FIGS. 9 and 10) the poly vinyl chloride sheets together to form a cuboid of the dimensions shown in FIGS. 9 and 10.

The cupid has a large diameter air inflation port FIGS. 9 and 10 Part B of between 12 millimeters and 21 millimeters inside diameter welded 60 millimeters from one vertex and on one of the external 13 inch×13 inch sides or faces of the cuboid.

There are five square internal I beams FIGS. 2, 9 and 10 Part D made of 0.45 thick poly vinyl chloride material cut square to the size of 13 inches×13 inches. The I beams have a 25 millimeter diameter holes cut in the middle of them Part F for air to pass through when inflating and deflating the cuboid. The I beams have rounded corners as shown in FIGS. 1, 2, 9 and 10. The I beams are radio frequency or high frequency welded along their straight edges to the exterior poly vinyl chloride sheet material. The I beams are not welded at their corners. The internal I beams Part D and the square 13 inch×13 inch surface face sheets Part E with rounded corners FIGS. 1, 9 and 10 are evenly spaced in the cuboid FIGS. 9 and 10 Parts D and E.

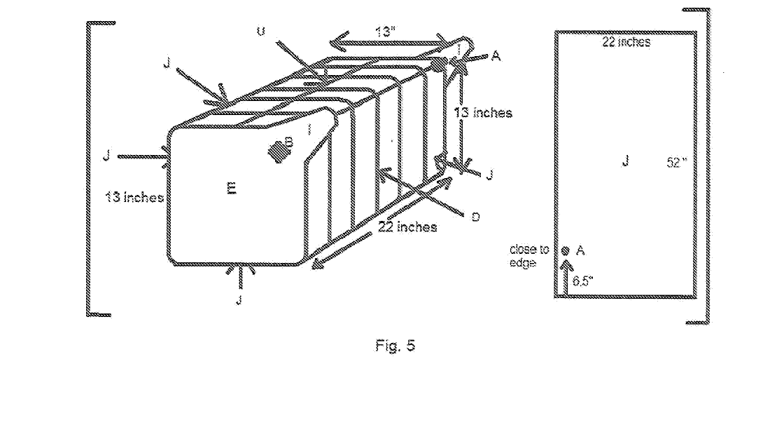

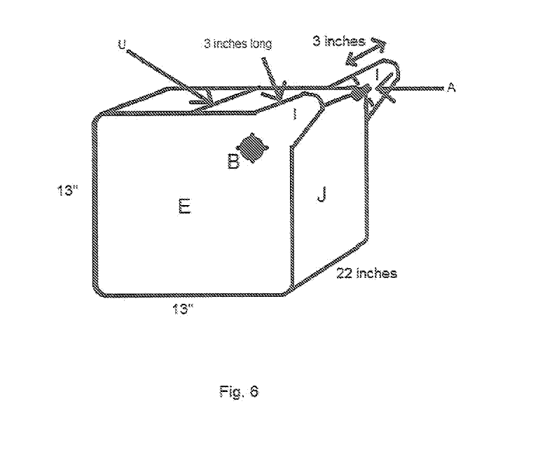

The cuboid has four rectangular sides or faces made from one piece of 0.45 millimeter thick poly vinyl chloride sheet material cut to the rectangular size of 22 inches×52 inches FIGS. 5, 7, 9 and 10 Part J; this sheet wraps around the I beams Part D and end sheets Part E and is welded only to each straight edge of the I beams Part D and welded all the way around the entire edges of the two 13 inch×13 inch sides or faces Part E which are made of 0.045 millimeter thick poly vinyl chloride. The 22 inch×52 inch sheet forms four faces of the cuboid each of which are 22 inches×13 inches rectangular in shape.

Air is injected into the cuboid through the large diameter air inflation port FIGS. 9 and 10 Part B. The air pushes the external sides or faces out to form a cuboidal shape. The air, along with the internal and surface structure provide the cuboid with the rigidity it needs to supply pull from leverage to induce spinal tradition when used on the edge of a bed, cot or table.

### All Models of Cuboids

The different construction, features and illustrations of the different models of the cuboid human spinal traction device other than and including the model being manufactured and offered for sale are described in the following text:

This Cuboid human spinal traction device is made as different models as a cube FIG. 22, a rectangular cuboid FIGS. 13-21 or a square prism (also known as a square cuboid) FIGS. 6, 8, 10, 27, 28, 32-34, 39-45, 52-54 for use on the edge of a sleeping bed FIGS. 29, 31, 49-51 or mattress, or table or any flat surface that has an edge and or vertical side or face that a human user can lie on horizontally. The cube, rectangular cuboid and square cuboid will be referred to as cuboid in the remainder of the text. Cuboid is the basic functional shape of the cuboid human spinal traction device, whether different size and shape models be made as a cube, rectangular cuboid or a square cuboid size and fitment purposes of different sizes and shapes of human users.

When the cuboid traction device is made as a cube, it has six sides or faces that are mostly square FIG. 22. When the cuboid is made as a rectangular cuboid it has six sides or faces that are mostly rectangles FIGS. 13-21. When the cuboid is made as a square cuboid it has four sides or faces that are mostly rectangles and two sides or faces that are mostly squares FIGS. 6, 8, 10, 27, 28, 32-34, 39-45, 52-54.

As either a cube, as rectangular cuboid or as a square cuboid, the cuboid spinal traction device retains the same cuboid functionality in ail size and shape models as a cuboid human spinal traction device on the edge of a bed. The basic difference between the cube, the rectangular cuboid and the square cuboid are that they are shaped and sized to fit different shapes and sizes of human users. The cube, rectangular cuboid and the square cuboid are three models of the same, cuboid shape claim for patent protection for use as a spinal traction device. The cuboid shape, in all cuboid models, works the same in application whether as a cube, a rectangular cuboid or a square cuboid. The cuboid shape and size ranges are basic, fundamental and core claims for patent protection for the different cuboid spinal traction models listed within this text.

When the cuboid is constructed as an inflatable and is inflated, parts of the external material of the cuboid bulge out in curved or rounded shapes but the general overall functional cuboid shape is retained. Different models of inflatable cuboids are made with internal structure of either I beams FIGS. 2, 5, 7, 9, 11, 12, 23, 26, 27, 32-46 Part D, tubes FIGS. 32-38 and 44 Part C, cubes FIGS. 27 and 28 Part L or any combination of these structures to better hold the overall outside cuboid shape while inflated. Some models of cuboids have I beams with large holes in them FIGS. 32, 36-38 and 44 Part CC for tube(s) to go through them. The internal structure is changed in different models to better fit different sizes and shapes of human users. In all inflatable models, the cuboids are inflated more for longer legs and inflated less for shorter legs of human users.

Some models have indentions FIGS. 23, 39-48 and 52-64 Part S on one side to better conform and contour to the legs and hold the legs in place. The indented models can be used by different sized individuals since the indentions effectively reduce the depth of the cuboid for a smaller individual. A larger individual can position the indented cuboid on the edge of the bed to where the indented side only touches air and does not contact the bed or the user. Adjusting the amount of inflation of these cuboids extends the range of the size of individuals who can comfortably and effectively use them.

Some models of cuboids have indentions FIGS. 45, 46 and 52 Part S on two sides or faces. These cuboids conform and contour to the legs and hold the legs in place. These cuboids can be placed on the edge of a bed for different ways for better fitment comfort and functionality for a greater range of size of individuals. Adjusting the amount of inflation of these cuboids extends the range of the size of individuals who can comfortably and effectively use them.

The cuboid has different cuboid shapes and sizes for fitment to different shapes and sizes of human bodies. The external overall cuboid size ranges in all models are from 8 inches×8 inches×8 inches outside dimensions up to and including 21 inches×21 inches×36 inches outside dimensions. The different overall size dimensions of all cuboid spinal traction device models are shown in FIGS. 13-22.

Some models of cuboids have friction or anti-skid material FIGS. 5-10 and 47 Part J of any type of material on four outside surface areas to better hold the cuboid in place on either the edge of the bed or table, the skin of the legs or the clothes on the legs of the user.

Some models of cuboid spinal traction devices are constructed of a material within the listed size ranges that forms a cuboidal shape for use on the edge of a bed or table for spinal traction to where the cuboid weighs less than 6 kilograms because the cuboid shape is the fundamental shape that provides spinal traction when the cuboid is used on the edge of a bed or table.

The different models of cuboid spinal traction devices have edges that are either pointed FIGS. 13-29, 31-36, 38-46 and 52 or rounded FIGS. 1-10, 2, 37, 53 and 54. Corners and vertexes are either pointed or rounded—see the illustrations in the previous sentence.

When the cuboid is constructed as an inflatable FIGS. 1-12, 23, 24, 26-28, and 32-46, it is made of poly vinyl chloride (pvc), poly vinyl chloride coated fabric, polyurethane coated fabric or any fabric or material or any combination of fabrics or materials that can be radio frequency welded (high frequency welded), glued, sewn or sealed in such a way that the cuboid can function for the purpose of the device and hold, capture and seal air inside the structure of the cuboid. As an inflatable the cuboid is welded or glued and sealed at the corners, at all external edges where two sheets come together, at the straight edges of the I beams D, at the ends of the tubes FIGS. 32-38 and 44 Part C, at the ends of the cubes FIGS. 27 and 28 Parts L and where edges of the external material Part J come together FIGS. 5-10 Part U and in FIGS. 4-10, 23, 26, 27, 32 39, 45 and 48 around the inflation, deflation ports B and in FIGS. 5-8 Part A.

As a non-inflatable, the cuboid FIGS. 13-22, 29-31, 49-54 are made from any material including all types of plastic either biological or synthetic to form a cuboid frame that is covered with any type foam or batting of the type that can slightly compress, is resilient, can hold the general shape of the cuboid, provides comfort and can function for the purpose of providing spinal traction while in use on the edge a bed; these models have a surface cover of any type. Models constructed of foam and or batting FIGS. 13-22, 29-31, 49-54 are made of any type of foam and or batting and or is biological or synthetic, is resilient, is compressible, and is firm enough to hold the shape of the cuboid in such a manner that it functions as a spinal traction device while in use on the edge of a bed. The foam and or batting constructed cuboids include a surface of any type including an impermeable outer surface of any material, including plastic, vinyl, or fabric, whether synthetic or biological. Some models of the cuboid of any size and construction have a permanent or replaceable cover FIG. 47 of any material which does or does not include an abrasive or anti-slip part side or face where it contacts the bed surface or the legs or clothes of the user.

Some models of cuboids have a protrusion or heel FIG. 25 Part M along one edge or corner to better keep the cuboids in place on the edge of the bed while in use.

Surfaces of some models of the cuboids FIGS. 23, 39-46 and 52-54 include contour shaping and or an indention Part S for more comfort and to contour to the legs.

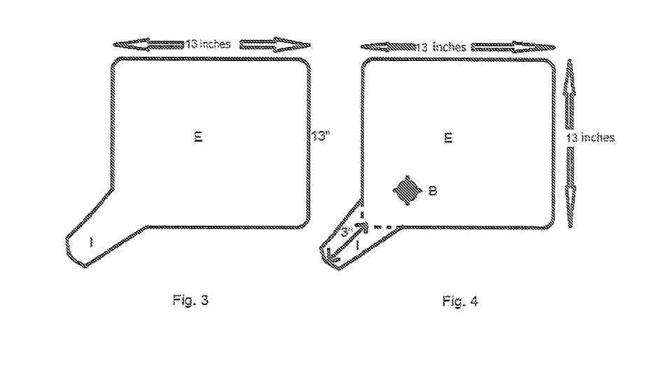

Some models of cuboids have tabs FIGS. 3-8 Part I and some models of cuboids have handles FIG. 29 Part P attached to the cuboid near to where the cuboid touches the upper leg near the buttocks for the user to more easily grasp, place and adjust the cuboid while the user is lying down.

Some models of cuboids have a sealable inflation and deflation tube FIG. 24 Part K attached to the cuboid for easier adjusting of the amount of inflation while in use by mouth and or by hand. Some models of cuboids have an inflation and deflation tube FIG. 24 Part K that has a hand operated inflation pump Part O with a valve for deflation.

The inflatable cuboid has a large internal diameter orifice sealable quick inflation and deflation port of any type or size FIGS. 4-10, 23, 25-27, 32-39, 45 and 46 Part B. This allows for quick inflation and deflation and saves time and energy while inflating and deflating. This allows the device to be more quickly and easily used after storage and stored after use. This feature ensures the device is more likely to be used when there is a desire to use it. The port is placed on one of the sides or faces E of the cuboid that does not contact the user or the bed and near or approximately 6 centimeters from a vertex of the cuboid that is both closest to the edge of the bed and the user's buttocks for easy access for inflation adjustments while in use.

Some models of cuboids have a 5 to 12 millimeter air inflation and deflation valve FIGS. 5-8 Part A with an attached plug for sealing, for making small inflation and deflation adjustments for functionality and for size fitment to the individual user. The small air inflation valve A is placed on one of the long rounded edges of the cuboid near a vertex so it does not contact the user or the bed. This placement of the small air valve allows for easy access for the user to make adjustments to the size and shape of the cuboid while in use.

Air is injected into the cuboid device through the large air port FIGS. 4-10 23, 25-27, 32-39, 45 and 46 Part B, the air tube FIG. 24 Part K and or the small air valve FIGS. 5-8 Part A to fill the cuboid to the optimum amount of inflation to provide structure, support, shape, cushion, comfort, compression and functionality.

In some models of the inflatable cuboid, I beams FIGS. 5-10, 23, 26, 27, 32-46 Parts D are attached to the insides of the sides or faces Part J to support the cuboid functional shape. The I beams keep the sides from bulging out too much when inflated and while in use. The I beams provide more contour shape to the legs and allow the cuboid to better compress on and deform on the edge of the bed. The I beams help keep the cuboid from sliding on the edge of the bed while in use.

Some models of cuboids have I beams Part D placed in different positions FIGS. 5-10, 23, 26, 27, 32-46 with as few as one I beam per cuboid or many as necessary.

Some models of cuboids FIG. 23, 26, 32-35, and 38-46 have different numbers of I beams placed in different positions or arrangement so the sides can round out or billow out in such manner that the rounded out or billowed out external sides or faces are more contoured to the legs for comfort and to better hold the legs in position.

Some models of cuboids FIGS. 23, 32-35 and 39-46 have different dimensions of I beams Part O that are welded to the outer sheet or sheets in such a way that the I beams cause indentions Part S in the cuboid to better hold the legs in position.

The I beams FIGS. 2, 7, 9, 12, 26-28, 32-40, 45 Part F have large holes Part F in them for inflation and deflation. The holes are large and of the size 20-25 millimeters to allow air to quickly pass through for quick easy inflation and deflation without substantially reducing structural integrity.

Some models have two tubes FIGS. 32-35 Parts C attached to the top side or face and the bottom side or face of the cuboid to form indentations for the purpose of conforming better to the shape of the lower legs. The tubes also help keep the side of the cuboid that contacts the edge of a bed from bulging out which could make the cuboid less stable. The tubes have holes Part F for inflation and deflation.

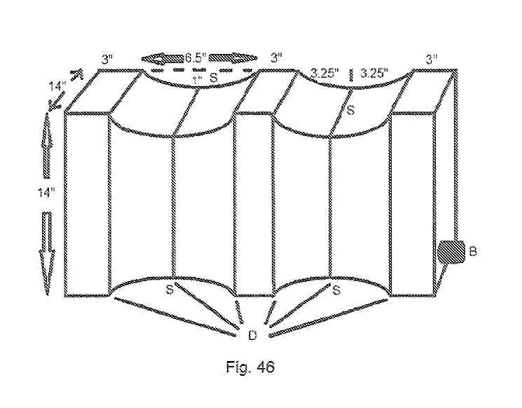

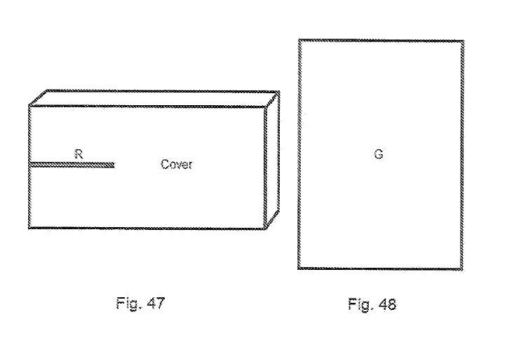

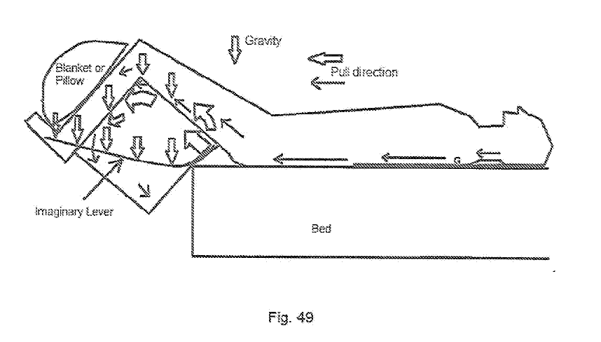

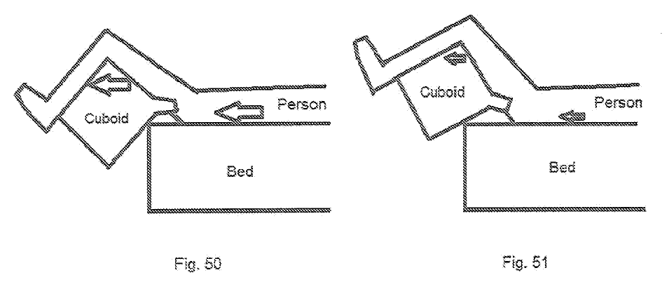

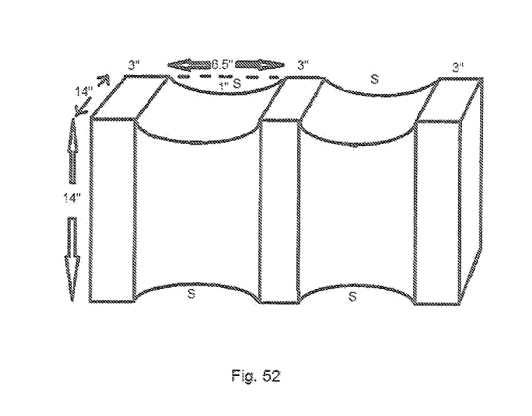

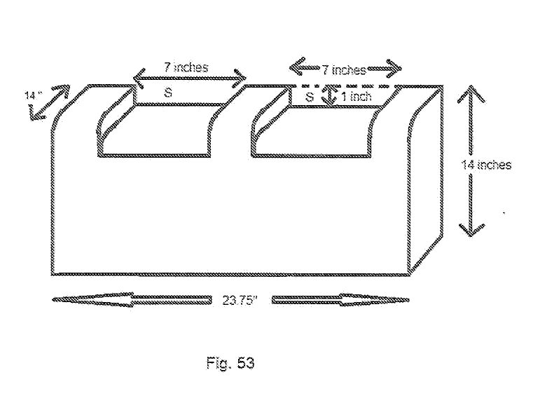

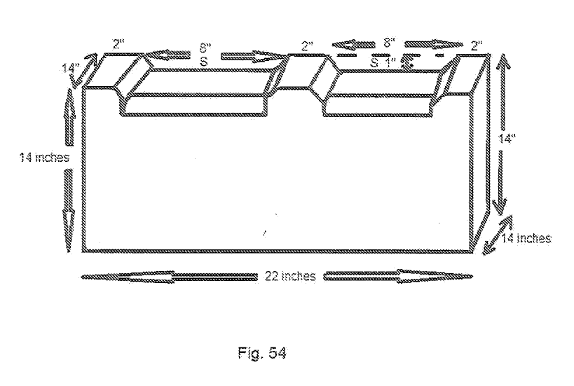

Some models of cuboids FIGS. 36-38 and 44 have an internal tube Part C that is the same length as the cuboid to hold the sides or faces in place and to keep the sides or faces from rounding or bulging out. The tube C has a hole F or holes in them for allowing air to pass through for inflation and deflation purposes. The holes F are of the size that they allow air to quickly pass through for quick and easy inflation and deflation without substantially reducing structural integrity.

Some models have a permanent or removable form fitting cover FIG. 47 with or without a zipper Part R for removal and installation. The covers are made of any suitable material to fit and cover the cuboid for easier cleaning. Some models of covers are made of materials that have anti-skid properties that make the cuboid have more friction between the user and the bed for the purpose of making the cuboid stay in place without skidding while in use.

Some models of cuboids include suitable sized sheets of thin anti-skid or anti-slip material or fabric FIG. 48 to be placed between the mattress, the mattress pad, and or the sheet under the cuboid on the edge of the bed to create friction to keep the cuboid from sliding on the edge of the bed.

Textured pvc coated non-skid fabric material and or thermoplastic polyurethane coated or any type of non-skid fabric, coating or any material of the type that sticks to or holds in place well to human skin, bed sheets or a table is used for the outer surfaces of some models of cuboids. These materials help spread out he pull from the cuboid over a greater surface area of the legs reducing localized pressure and increasing comfort. These fabrics or materials help hold the cuboid in place on the bed sheet and on the edge of the bed and or mattress. These anti-skid materials help keep the cuboid from sliding on the edge of the bed.

Some models include appropriate sized sheets of textured poly vinyl chloride coated non-skid fabric or thermoplastic polyurethane coated fabric or any non-skid fabric or material FIG. 48 is placed between the mattress and the mattress pad, between the mattress pad and the sheet, and between the sheet and the head and upper torso of the user for the purpose of creating friction between the user and the bed. This material helps keep the user from sliding on the bed or table while traction is applied.

Some models of cuboids have a sheet of material FIGS. 30 and 31 Part Q extending from one edge or seam on a face of the cuboid out a number of inches and long enough so the sheet is placed under the pelvis of the human user while the cuboid is used for spinal traction on the edge of a bed or table. The user lying on this extension sheet helps hold the cuboid in place while in use.

Some models of the cuboid are welded only in the middle of the I beams on only one or two sides or faces of the cuboid FIGS. 11 and 12 Part H. The surface is not welded to the I beam edges Part W. This allows the external side or face sheet to round out or billow out Part V in such a manner that the face sheet better conforms or contours to the user's legs. This increases comfort because pressure is applied to more surface area of the legs.

### Discoveries and Inventions

### How the Cuboid Provides Spinal Traction:

The user lays on his or her back on a bed with his or her buttocks near the edge of the bed FIGS. 49, 50 and 51. One flat side or face of the cuboid is placed on the edge of the bed at a 45 degree angle to the top surface of the bed, with one edge of the cuboid pointing up FIGS. 45 and 46. The user places both of his or her legs over the top of the cuboid with the knees directly over the top edge of the cuboid. The side or face of the cuboid touching the edge of the bed compresses against the edge of the bed as the legs are relaxed. The weight of the relaxed legs forces the top of the cuboid to rotate away from the bed. The cuboid and the edge of the bed compress at this same time. The rotation and compression of the cuboid and the bed applies a push on the legs away from the bed. The legs pull on the pelvis, which pulls on the spine and induces spinal traction especially on the lower spine (lumbar section of the spine). The traction is facilitated because the head, arms, neck and upper back are held in place on the bed by gravity and friction, while the legs, pelvis and spine are pushed by the cuboid in the direction away from the user's chest, neck and head. With all of he muscles in the entire body of the user completely relaxed, the cuboid stops rotating, is held in place by the compressed face of the cuboid, the compressed edge of the bed and by the user's relaxed lower legs. After the cube stops rotating, and is held in place, the cuboid applies a constant sustained pull for spinal traction.

The amount of pull is completely adjustable FIGS. 50 and 51 from zero pull up to maximum, simply by moving the cuboid up or down on the edge of the bed and by the user lying closer to or farther from the edge of the bed. FIG. 50 is more pull and FIG. 51 is less pull. Maximum pull is determined by the weight of the legs and the weight of the head, neck, arms and upper torso of the person using the device. The user has total control over the amount of pull and traction applied. Blankets or a pillow are laid on the feet of the user to increase the pull and traction FIG. 49. If the user lies far enough from the edge of the bed with legs over the cuboid, there will be zero pull FIG. 51.

When in use as a human spinal traction device, the cuboid is a class 1 lever FIGS. 49-51. The edge of the bed is the fulcrum. The weight of the legs and gravity supply the force. Whenever a force is applied to an object that causes that input force point of the object to move farther than the output force point of the object, the output force point of the object in increased over the input force point. Because the corner of the cuboid near the feet is farthest from the fulcrum (the edge of the bed), this corner moves the most under the weight of the legs than all the rest of the cuboid. There is more output force against the legs because of the lever action of the cuboid than the input force from the weight of the legs. Because part of the leg and the foot extend out farther from the fulcrum than the cuboid, there is amplified leverage force applied to the entire surface of the cuboid that touches and pushes on the legs.

The inertia of the human body and the friction of the body on the bed create the resistance. Because of the inherent shapes of the cuboid and the human body, the cuboid supplies forces in multiple directions to the legs of the human body but always away from the bed FIGS. 49 and 50. The forces are always applied in combination of the directions of up and away from the bed and down and away from the bed. There are three primary directions the cuboid supplies force to the human legs. The primary and strongest force is applied to the lower legs at the knee by the topmost part of the cuboid; this force pushes the lower leg mostly away from the bed but also down in an arc. The second primary force is applied just below the buttocks to the upper femurs and hamstrings pushing them up and away from the bed. The third primary force is applied along the entire contact surface of the cuboid and the upper legs. This force pulls the upper leg away from the bed. The combination of these multiple directional forces pulling over large surface areas of the legs spreads out the pulling force from leverage and reduces localized pressure to comfortable levels.

Whenever a force is applied to a smaller area, the pressure per square inch goes up. Whenever a force is applied to a larger area, the pressure per square inch goes down. The head, neck, shoulders, arms and the upper torso pressing down against the bed over a large surface area spreads the force, from the pull from the cuboid against the friction and resistance of the human body against the bed, over a large surface area of the human body. This large surface area of the body receiving friction and resistance against the pull of the cuboid, reduces localized pressure from friction and resistance to comfortable levels.

The inflatable cuboid conforms and contours to the shape of the human leas very well because the surface of the cuboid is so flexible. This contouring action causes the cuboid to contact more surface area of the legs which further reduces localized pressure and increases comfort. Not having the cuboid fully inflated increases surface area contact and comfort.

The combination of comfortable pulling from the cuboid on the legs and comfortable friction and resistance from the bed supplies comfortable traction.

The inflatable cuboid is more comfortable temperature-wise against the skin. Because the surface area of the inflatable cuboid is so thin and because the cuboid is filled with only air, the cuboid has much less mass. Because the inflatable cuboid has less mass, when the human skin initially touches the cuboid, heat transfer from the skirt to the legs is minimal. Therefore, the inflatable cuboid does not cause an uncomfortable cold shock upon contact or a continual cold feeling.

The cuboid works best when the surface has anti-skid qualities.

The cuboid works best applying traction when the user's upper torso's skin is in direct contact with the bed sheet or a thin sheet of anti-slip material. The cuboid works best when the user has bare skin or pants that do not slip touching the cuboid.

When the cuboid is providing traction to the spine, the user will feel a pulling of the skin on the middle and upper back of the middle and upper torso, on the back of the head and sometimes on the back of the neck. It is not necessary initially to feel anything in the spine to know traction is happening. Some users feel the traction on their spine immediately and others do not. After being in traction for two to six minutes, it is normal to start feel stretching of the muscles of the lower back and decompression of the spine. The user allows the small amount of time he or she is in traction to slowly stretch the muscles of the lower back. As the muscles are stretched, the vertebrae of the lower back are pulled slightly apart decompressing the disc material between the vertebrae. It is possible this decompression can reduce disc pressure on nerves causing pain. It is possible a decompressed disc can heal over time back to or close to a former healthier shape and condition.

The overall external size and shape of the cuboid FIGS. 13-22 when placed in its usable position on the edge of a bed and when a person is laying on his or her back on the bed with legs placed over the top of the cuboid FIGS. 49 and 51 causes the cuboid device to rotate a certain amount, with the top corner moving away from the bed and user. This rotation causes the cuboid to pull on the human legs which pull on the pelvis and spine inducing spinal traction. After the cuboid causes a constant pull, the cuboid stops rotating and is held in place by its shape, the shape and resistance of the totally relaxed legs and the edge of the bed. The weight of the human body's friction against the bed also stops the cuboid from rotating more, stops the cuboid's rotation and holds the pull and the traction constant. The cuboid does not fall off of the bed for the above mentioned reasons and also because the cuboid's surface curves against the bed helps to hold it in place. The anti-friction quality of the material on the cuboid helps hold it in place. The heel on some models help hold the cuboid in place.

The amount of pull and traction is completely adjustable from zero pull and traction up to maximum pull and traction. The lighter the weight of the user's legs, the less maximum pull. The heavier the weight of the user legs, the more maximum pull. The user is in total charge of and control of the amount of pull and traction created. Because the cuboid is a class 1 lever, the placement of the cuboid either higher or lower on the edge of a bed extends or retracts the length of the cuboid that is out and away from the fulcrum of the edge of the bed and adjusts how much pull and traction the cuboid lever applies to the legs and the spine. The user can also move closer to or farther away from the edge of the bed to increase or decrease the amount of pull the device applies to the spine. Moving the body farther away from the edge of the bed puts less weight of the legs out beyond the edge of the bed which reduces pull from the cuboid. Moving the body closer to the edge of the bed puts more leg weight out over the cuboid and away from the bed increasing pull and traction.

A pillow or folded blanket are laid over the feet of the user to create more pull. Pillows or blankets are placed over the arms and chest of the user to hold the upper torso in place so the upper torso doesn't slide toward the device which reduces the amount of pull. Pillows and or blankets are for the majority of the time not necessary except in cases where a user may be very light weight and more pull and or upper torso friction on the bed is needed.

The user can increase the pull of the device by tightening up the stomach muscles and buttock muscles and lifting the pelvis slightly. This action allows the cuboid to rotate a little more away from the bed. After this, the user relaxes all muscles of the body and the cuboid is applying more pull since it has rotated a little more.

The user can increase or decrease the amount of pull and traction by adjusting the amount of air in the inflatable cuboid. Because the user places the cuboid on the edge of the bed with the air inflation port or air inflation tube close to the edge of the bed, the air inflation port or inflation tube is accessible for inflation adjustments.

Shorter users inflate the inflatable cuboid to a less full amount. This allows the cuboid to deform a degree to fit their size while still maintaining full functionality. It is best for all users to adjust the amount of inflation to be the most comfortable and effective for them. It is usually not necessary to fully inflate the cuboid to its fullest shape. Taller users may find a more full inflation provides the best results for them but not necessarily. It is usually not necessary to inflate the cuboid to where it has even a small amount of pressure pushing outwardly when not in use. The cuboid is a very low pressure device even when in use because the pressure is spread out over so many cubic and square inches. It does not take much pressure to hold up human legs or to supply the necessary pull for traction when the force necessary is spread out over such large surface areas. The internal air pressure is spread out over so many square inches that the pressure is very low when in use. The inflatable cuboid material is thin but more than adequately strong.

Since the cuboid is constructed as an inflatable, out of foam or with b batting, it is compressible. Since it is compressible, it create pressure pushing outwardly when in use. This outward pressure helps push the legs away from the body. This helps create traction of the spine. Since the device is placed on the edge of a sleeping bed, the edge of the bed compresses and pushes up and away from the bed and the human torso. This helps push the legs away from the torso creating traction on the spine. Even though these factors help apply a pull and traction, the cuboid is moved up on the edge of the bed and the human body is moved a little farther away from the edge of the bed to create very small amounts of pull including all the way down to zero pull and zero traction. Very small amounts of pull and traction are used for longer periods of time to gently hold the spine in place so too much stretching of the muscles and too much decompression of the disc material does not take place. This can possibly facilitate longer periods of possible reduced or possible eliminated pain possibly from herniated or bulging discs.

The length, width and height of the cuboid are different for different size models for different size and shape individual users FIG. 13-22. Different models are made as cubes, rectangular cuboids or square cuboids. However, the same general overall cuboid shape is retained to function as a human spinal traction device while used on the edge of a bed. The I beams and tubes in the inflatable cuboids are put in different places inside the cuboids to make different models for different size and shaped individuals. However, the same general overall cuboidal shape is maintained in the different size models. The same principals of how the cuboid uses friction, gravity, leverage, rotation, compression and incorporates a bed, and the human body to achieve relaxed, comfortable spinal traction remains the same in all models. The same ways to adjust the amount of pull and traction is maintained in all models.

A main difference between the inflatable cuboids and other models of cuboid human spinal traction devices is the inflatable cuboids incorporate the amount of inflation and deflation to adjust comfort, fitment, the amount of pull and the amount of traction. The other differences are that the inflatable cuboid is deflated to be stored and transported in a fraction of the space of other models cuboids.

The upper side or face of the cuboid under the hamstrings of the upper legs, closer to the buttocks and the corner of the cuboid that is pointing at the human body FIGS. 49-51 rotate up a small amount as the cuboid rotates. This small upward but strong rotation lifts the legs, the buttocks and the pelvis up a little where they are not pressing as firmly on the top of the bed. This decreases the amount of friction between the buttocks and the bed. This allows more pull from the cuboid device to be applied to the spine. This lifting of the legs and pelvis does not change the amount of friction and gravitational pull on the upper torso, neck, head and arms against the bed. Because the head, neck and upper torso stay in place on the bed the entire spine is in traction. However, the strongest pull is on the lower spine, where the vertebrae and discs are the largest and strongest.

For users with certain disc problems (possibly like anterior bulging disc material protruding out from between the lumbar vertebrae), a rolled up towel or a small pillow is placed under the small of the lower back while using the cuboid for traction. The pillow induces or supports the normal inward curvature of the lower spine during traction. This possibly may allow the traction to stretch the anterior edges of the vertebrae more apart allowing the possibility for the anterior disc material to be suctioned back in to where it is not bulging out as much or is possibly suctioned back in between the vertebrae. If suctioned back in place over time, disc material possibly may have the opportunity to heal more between the vertebrae or possibly back where it belongs and away from nerves.

The cuboid device can be used in conjunction with pieces of anti-slip material 48 Part G similar to and including rubberized or rubber like material placed on the mattress, on the mattress pad and on the bed sheet directly under the head and upper torso of the user so the head and upper torso stay in place while the legs, pelvis and spine are being pulled. The anti-slip material is not placed under the lower back or pelvis because the pelvis and lower back need less friction so they can be pulled in the direction toward the cuboid and away from the upper torso of the user.

The best time to use the device is just before going to sleep for six to eight hours and or for the night. After the spine has been decompressed and the muscles stretched, lying in bed in a neutral spinal condition, or in other words a non-compressed and not in traction spinal condition, provides time for possible reduction of swelling, possible healing and possible pain reduction. It is also possible that with the vertebrae possibly slightly farther apart while lying down, especially after having been in traction, as compared to when spinal discs are compressed from a body being vertical, is when blood, oxygen and nutrients have more space and time to reach damaged or inflamed areas of the spine.

A convenient advantage of using the cuboid device to decompress the spine, for a short amount of time, immediately before going to sleep for an extended period of time, is that the user is already on his or her bed. Because of where the cuboid is used, it is more likely to be used more often.

With the device being quickly and easily deflated to fit in such a small space, it easily and safely can be stored on a bed stand, under a bed, in a drawer, in a closet or in luggage.

Because the cuboid is quickly and easily deflated to fit into such a small space, it can easily be transported in luggage, while adding very little weight, for use away from home.

With the cuboid being so light when inflated or deflated, it puts little or no strain on a person who has to pick it up, move it or position it for use when they are experiencing back issues and or pain.

The cuboid is very ergonomic and easy to discontinue the traction session since the user is not down on the floor, or strapped in anything or away from the comfort of a bed. The user simply has to move legs out to the side or lift them up a small distance and allow the cuboid to drop to the floor while the user's buttocks, lower back, torso neck, head and arms are still lying on the bed. The user is always on the safety of his or her bed. The user can sit up on the bed after the traction session and since the cuboid is tall and light it is easy to pick up to be moves to a safe place or quickly deflated for storage.

Since the human body is not strapped to or placed in but rather only lying on the cuboid traction device on a bed, the human body itself regulates how much traction is applied to the spine. The muscles, tendons, cartilage, vertebrae and discs of the human body keep the body together and unitized. The human body resist the pull from any traction devices. If the pull of the cuboid should ever exceed the amount the human body's make up predetermines may be excessive, the composition of the human body allows the human body to slide on the bed toward the cuboid keeping the pull from the cuboid from becoming excessive. Also, the lower legs tend to straighten out with more pull which also helps keep the cuboid from pulling too much.

The use can also control the amount of traction applied to the spine by contracting or tightening the muscles of the back, stomach and sides of the abdomen. The tightening or contracting of these muscles while in traction from the cuboid is a spinal compressing action and reduces the amount of pull and the amount of traction applied to the spine. The tightening of these muscles can be done while repositioning the cuboid or the human body. The user can tighten these muscles so the user can scoot away from the edge of the bed slightly which will also reduce the amount of pull from the cuboid. It is good to learn control of these muscles for use while in traction if flexing these muscles are ever beneficial while in traction. Tightening these muscles reduces the traction applied to the spine.

### More Precise Use of the Cuboid

Exercises to Experiment with while in Traction:

The user can tighten either the right side abdominal muscles, the left side abdominal muscles, the right side muscles above the hips, the left side muscles above the hips, the right side back muscles or the left side back muscles or a combination of right or left muscles while leaving all of the other muscles of the body relaxed. This action can induce a torque and can direct the traction to a specific location and side of the lower spine. This can possibly help realignment of the lower spine. This can possibly induce a stretching and or a suction to a specific problematic area of the lower spine, it is recommended that the user take charge of learning muscle control and the use of the traction cuboid to find what amount of pull and what actions of the user provides the most comfort, possible realignment, possible pain relief and possible healing. Once the most comfortable position is found, the muscles can be relaxed. Most of the time, the user is completely relaxed while traction is applied

### Other Uses for the Cuboid:

The cuboid can be placed on one of its flat sides or faces on top of a bed. A person can be lying on their back on their bed with their buttocks close to a vertical side of the cuboid and with their lower legs resting on the top flat surface of the cuboid. If positioned correctly, the legs can give a very slight pull on the pelvis up and away from the body of the user, causing a small amount of traction applied to the spine. This way the device can be used as a maintenance device for times when the spine is not as noticeably problematic.

For people who have swelling of the legs, the cuboid can be used as mention in the paragraph above. This use of the cuboid could possibly help reduce swelling of the legs. The cuboid can be inflated to the most comfortable shape for this purpose to reduce pressure and increase blood flow.

The cuboid could be used on top of a bed lying flat on one of its sides or faces. A user could lay on his or her back in bed and place one lower leg on top of the cuboid in such a way that it would tilt the pelvis. For certain inflammation, soreness, bulging disc or strained muscle on one side of the spine, this could possibly provide more comfortable positioning and possible relief.

The cuboid device can be used as a foot rest while sitting in a stable comfortable arm chair. This, in some cases, may possibly be more comfortable for some spinal conditions. The elevated position of the feet and legs could help with vital blood circulation and could help reduce swelling.

1. The cuboid human spinal traction device: is made in the shape of a cuboid or in the basic overall shape of a cuboid; is made as models with a rectangular cuboid shape or the basic overall shape of a rectangular cuboid; is made as models with a square cuboid shape or the basic overall shape of a square cuboid; is made as models with a cube shape or the basic overall shape of a cube; is made as models in different sizes within a certain described size range; has rounded or pointed corners and vertexes; has parts that bulge out or form rounded or curved surfaces; is used on the edge of a sleeping bed, mattress, cot or table; acts as a class one lever; uses the weight of the legs to supply power for leverage and for the cuboid to rotate and pull on the legs which pull on the pelvis which pulls on the spine inducing spinal traction while the head, neck, arms, shoulders and torso are held in place on the bed, cot or table by gravity and friction; is made as inflatable models with many different described internal and external structures; is made or shaped as model made from and or consisting of firm, compressible foam and or cushion material in described sizes and shapes; is made as models constructed with a frame made of and or from different described materials in different described sizes and shapes which are covered with batting, foam or cushion material, with a sewn, molded or any type of surface or cover; is made as models with a described protrusion or heel at one corner and edge of the cuboid to help hold the cuboid in place on the edge of a bed, cot or table; is made as models with described indentions on one or two sides or faces of the cuboid to better fit, conform or contour to the shape of the legs; is made as models with tabs or handles for grasping with hands for positioning and adjustments while in use; is made as inflatable models in described construction, sizes and shapes with inflation and deflation port(s) and or valve(s); is made in inflatable models with an attached long sealable inflation tube for ease of inflation or deflation while in use; is made as inflatable models with a long inflation tube with an attached hand pump for ease of inflation and a valve for ease of deflation while in use; is made as models with a pliable sheet of material that is attached to and extends from the surface of the cuboid and this sheet is placed under the pelvis of the user so the pelvis can help hold the cuboid in place and help keep the cuboid from sliding on the edge of the bed, cot or table; is made as models with an anti-skid and or anti-slide surface; is made as models with surface material that has anti-skid and or anti-slide qualities; is made as models with surfaces that have resistance to viruses, bacteria and fungus; is made as models that have impermeable surfaces to biological matter.
